(19) United States
(12) Patent Application Publication
(10) Pub. No.: US 2010/0216233 A1
(43) Pub. Date: Aug. 26, 2010

(54) ATTENUATED INVASIVE E.COLI STRAINS AND APPLICATIONS THEREOF AS INTRACELLULAR VECTOR FOR THERAPEUTIC MOLECULE

(76) Inventor: Catherine Liliane Andree GRILLOT-COURVALIN, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/681,085

(22) PCT Filed: Oct. 2, 2008
(86) PCT No.: PCT/EP08/63262

§ 371 Date: May 6, 2010

(30) Foreign Application Priority Data
Oct. 2, 2007 (EP) 07301425.0

(51) Int. Cl. C12N 5/10; C12N 1/21; C12N 1/19

(52) U.S. Cl. 435/325; 435/252.33; 435/254.2; 435/419

(57) ABSTRACT

The invention relates to a vectorial system capable of delivering a nucleic acid of interest into eukaryotic target cells, said vectorial system including a recombinant non-pathogenic and non-replicative Escherichia coli bacterium (E. coli) having integrated into its chromosome, in a targeted manner and without any antibiotic marker, one or more genes imparting to said E. coli bacteria the capacity to penetrate into the cytoplasm of said eukaryotic target cells and to lyse the penetration vacuole. The present invention also relates to the use of such E. coli bacteria for the production of therapeutic compositions and their delivery without any purification step for preventing or treating diseases by vaccination or gene therapy.

The present invention relates to a vectorial system capable of delivering a nucleic acid of interest in eukaryotic target cells, or coding for a protein, this vectorial system comprising a recombinant non-pathogenic bacterium Escherichia coli (E. coli) having integrated into its chromosome in a targeted manner and without any antibiotic marker one or more genes imparting to said E. coli bacterium the capacity of penetrating into the cytoplasm of these eukaryotic target cells. The present invention also comprises the use of such bacteria E. coli for delivering therapeutic compositions intended for preventing or treating a disease by vaccination or gene therapy.

The transfer of genetic material in mammalian cells by means of a non-pathogenic invasive E. coli bacterium has already been the subject of publications (French patent application FR 95 15556 published under number 2 743 086; Grillot-Courvalin C., Goussard S., Huetz F., Ojeius D. M., and Courvalin P. (1998), Nature Biotechnol., 16:862-866).

The techniques described in these documents relate to a genetically modified E. coli bacterium with the purpose of penetrating and reaching the cytoplasm of determined target eukaryotic cells, of preventing its multiplication and its survival in target cells, as soon as it enters into the cytoplasm of these target eukaryotic cells. This bacterium is further transformed by a plasmid coding for a gene, for which transfer into the target cell is desired, which is a prokaryotic-eukaryotic shuttle vector, which may be replicative in said bacterium E. coli or further integrative in said target host eukaryotic cells.

More specifically, these documents describe such E. coli bacteria in which the genes imparting to the E. coli bacterium its capacity of penetrating and reaching the cytoplasm of target eukaryotic cells, such as the invasion gene inv of Yersinia pseudotuberculosis (Y. pseudotuberculosis), and the gene hly coding for the hemolysin of Listeria monocytogenes (L. monocytogenes) are brought by plasmids (of the pGB2Ωinv-hly type). Among the thereby transformed non-pathogenic and invasive recombinant E. coli bacteria, the strain E. coli BM2710/pGB2Ωinv-hly is particularly described (see also Courvalin et al., CR Acad. Sci., 1995, 318, pages 1207-1212). This strain BM2710 was deposited at the CNCM (Collection Nationale de Cultures de Microorganismes, Institut Pasteur, 25 Rue du Docteur Roux, 75724 Paris Cédex 15, France), on Oct. 27, 1995 under number 1-1635.

This initial work gave the possibility of showing that the strain of E. coli BM2710, auxotrophic for diaminopimelic acid (which is lyzed without being divided by lack of synthesis of its wall in a dap-deficient environment as this is the case in eukaryotic cells) and made invasive, was capable of delivering plasmid DNA to different cell types in which the expression of the transgene was able to be detected (Courvalin et al., 1995). For efficient transfer, two steps appeared to be necessary: the entry of the bacterium into the cell and its exit (or that of the plasmid DNA) from the induced phagocytosis vacuole. The internalization of the bacterial vector in many cell types was made possible by introducing into E. coli BM2710 dap, the inv gene of Y. pseudotuberculosis. This gene supervises the synthesis of invasin which binds with high affinity to receptors of the cell membrane, the β1-integrins. The exit from the phagocytosis vacuole is controlled by the acquisition by E. coli MB2710 of the hly of L. monocytogenes which supervises the synthesis of listeriolysin O. Listeriolysin O enables E. coli or its plasmid contents in the case of lysis of the bacterium, to exit from the phagocytosis vacuole by formation of membrane pores. In this first generation of the bacterial vector, the genes inv and hly were cloned in the plasmid pGB2 with a low number of copies which imparts resistance to spectinomycin and to streptomycin. It was shown that this bacterial vector gives the possibility by abortive invasion, of transferring and of having integrative or replicative vectors expressed by mammalian cells with good efficiency (5-20% of the cells depending on the in vitro transfected cell type: HeLa, CHO or COS-1 cells). The transfer efficiency of the plasmid of interest is evaluated with a reporter gene coding for the green fluorescent protein (GFP) placed under the control of an eukaryotic promoter. The transfer is also observed, but with a lower efficiency, in the absence of cell division or when the invasion is accomplished with cell confluence (Grillot-Courvalin et al., 1998).

The advantages of gene transfer by abortive bacterial invasion lie in the low toxicity for the receptor cells, the rarity of rearrangements in the delivered DNA, good transfection efficiency and also in the fact that the non-pathogenic and genetically well-defined donor bacterium is incapable of multiplying and even surviving in the receptor cell and in the environment because of autotrophy for diaminopimelic acid.

Among the improvements to be provided, mention may notably be made of the possibility of introducing any expression plasmid vector without having to observe the compatibility rules of the replication origins and also of the possibility of reducing or even suppressing the genes of resistance to antibiotics present in the vector bacterium.

The inventors have shown that it was possible to obtain such improvements by integrating into the bacterial chromosome genes imparting to the E. coli bacterium its capacity of penetrating and reaching the cytoplasm of target eukaryotic cells, such as the genes inv and hly.

The inventors have thus generated an E. coli strain which harbors both of these genes in the chromosome.

Apart from the aforementioned advantages, this chromosomal integration allows stabilization of the inv and hly genes in the progeny.

The object of the present invention is therefore a vectorial system capable of delivering in eukaryotic target cells a nucleic acid of interest or coding for a protein of interest, a vectorial system comprising a non-pathogenic recombinant E. coli bacterium, said non-pathogenic E. coli bacterium being further:
modified by introduction (by allele exchange) of one or more genes imparting to this non-pathogenic recombinant E. coli bacterium the capacity of penetrating into the cytoplasm of said eukaryotic target cells;
incapable of surviving in the cytoplasm of said target cell; and
modified by foreign DNA fragments,
   characterized in that the gene(s) imparting to said non-pathogenic recombinant E. coli bacterium, the capacity of penetrating into the cytoplasm of said eukaryotic target cells, are integrated into the chromosome of said non-pathogenic E. coli.

The expressions “vectorial system” and “target cells” reflect that the E. coli strain is used here as a means for transferring the nucleic acid of interest into said eukaryotic cells and not as a host bacterium for the expression of said DNA fragment, this may if necessary take place in said host cells after penetration of the bacterium into the cytoplasm. This vectorial system also covers the recombinant strains of E. coli such as those defined as a vectorial system and defined in the present invention.

By “nucleic acid of interest” is meant to designate here any nucleotide, DNA or RNA sequence either including or not enzyme restriction sites. Most of the systems for transferring genetic material do not allow transfer of large DNA fragments, viral vectors cannot accommodate fragments of more than 150 kb; by lipofection, large amounts of these large fragments have first to be produced from cultures of bacteria which replicate these bacterial artificial chromosomes, and then the DNA has to be purified and introduced into the cells by lipofection, all these steps often causing denaturation of the genetic material and resulting in low transfection efficiency.

Preferentially, the bacterial vector allows direct propagation and transfer into the cytoplasm of the cells, of these bacterial artificial chromosomes of any sizes without any preliminary purification and therefore retaining their physical integrity.

Thus, in an embodiment which is also preferred, the nucleic acid of interest may be of any size up to 100 kb or from 100-150 kb but also larger than 150 kb, the upper limit not being defined.

In a particularly preferred embodiment, the nucleic acid comprises at least 150 kb.

By “nucleic acid of interest” is understood here as a preference for any nucleotide sequence heterologous to the host cell or for any nucleotide sequence present in the host cell but the expression of which is desirably modified or regulated. Thus, preferably, this may be an exogenous nucleic acid.

In a preferred embodiment, the vectorial system according to the invention is characterized in that said non-pathogenic E. coli bacterium is transformed by chromosomal integration of two genes, stemming from one or two other bacteria, imparting to it the capacity of penetrating into the cytoplasm of said host cells.

In a preferred embodiment, the vectorial system according to the invention is characterized in that the gene(s) integrated into the chromosome of the non-pathogenic E. coli bacterium and imparting to it a capacity of penetrating into the cytoplasm of said host cells, allowing it to lyze the membrane of the vacuoles of said host cells.

When the control specificity towards said particular host cells is imparted by a gene stemming from another bacterium coding for the capacity of specifically penetrating said target cells, it is the selection of the gene responsible for the recognition and penetration of the cell membrane which imparts the specificity of the control. Said target eukaryotic cells may be animal cells, notably cells of mammals, in particular human cells or cells of yeasts or further plant cells.

The nature of the original bacterium of the gene responsible for the recognition and specific penetration into the determined target cells, used according to the invention, imparts to the vectorial system of the invention, a specificity of control towards a type of target eukaryotic cells, in particular cells of mammals, notably human cells, corresponding to the cell host spectrum of said bacterium.

Among the genes imparting to the bacteria the capacity of penetrating into certain host cells, mention may be made of:
a gene or a set of genes of the bacterium Salmonella typhimurium for transferring DNA into lymphoid tissue cells and hepatic cells;
a gene or a set of genes of the bacterium Shigella flexneri for transferring DNA into epithelial cells of mammals;
a gene of the bacterium of the Legionella genus, notably Legionella pneumophila, notably for transferring DNA more specifically into epithelial cells of the lung;
a gene of the bacterium L. monocytogenes for transferring DNA into epithelial cells of the central nervous system;
a gene of the bacterium of the Yersinia genus, notably Y. pseudotuberculosis or Y. enterocolitica for transferring DNA into epithelial cells; and
a gene of the bacterium of the Mycobacterium genus, notably Mycobacterium tuberculosis, Avilum complex, scrofulaceum for transferring DNA into macrophages.

In an embodiment illustrating this aspect of the invention, it is preferred to transform the E. coli bacterium according to the invention by two exogenous genes stemming from different bacteria. In particular, the invasion gene of the bacterium Y. pseudotuberculosis may be used in order to impart the capacity of entering the cells. The gene of the hemolysin of L. monocytogenes, a gene of about 2 kb, which imparts the capacity of intracellular dissemination by lyzing the membranes of the vacuoles, may also be used.

In a preferred embodiment, the vectorial system according to the invention, is characterized in that said non-pathogenic E. coli bacterium is transformed by chromosomal integration by the invasion gene of the bacterium or Y. pseudotuberculosis which imparts the property of penetrating into the cytoplasm of epithelial cells.

In a preferred embodiment, the vectorial system according to the invention is characterized in that said non-pathogenic E. coli bacterium is transformed by chromosomal integration by the gene coding for the hemolysin of L. monocytogenes or another pathogenic E. coli which imparts the property of lyzing the membranes of the vacuoles of said target cells.

The function of recognition and penetration of the cell membrane and the function for lyzing the membranes of vacuoles or phagosomes, the latter allowing the cytoplasm to be reached, may be imparted by one or more distinct genes, notably two distinct genes.

In a preferred embodiment, the vectorial system according to the invention is characterized in that said non-pathogenic E. coli bacterium is transformed by chromosomal integration both by the invasion gene of the bacterium Y. pseudotuberculosis which imparts the property of penetrating into the cytoplasm of epithelial cells and by the gene coding for the hemolysin of L. monocytogenes, which imparts the property of lyzing the membranes of the vacuoles of said target cells.

In a preferred embodiment, the vectorial system according to the invention is characterized in that said non-pathogenic E. coli bacterium is transformed by chromosomal integration both by:
1. the invasion gene inv of Yersinia pseudotuberculosis, preferably of sequence SEQ ID NO: 10 or of a sequence identical to at least 80% thereof and capable of imparting the property of penetrating into the cytoplasm of epithelial cells;
2. The gene hly coding for the hemolysin of Listeria monocytogenes, preferably of sequence SEQ ID NO: 4 or of a sequence identical to at least 80% thereof, and capable of imparting the property of lyzing the membranes of the vacuoles.

In a preferred embodiment, the vectorial system according to the invention is characterized in that:
1. the invasion gene inv of Yersinia pseudotuberculosis is under the control of the promoter Ptrc; and/or
2. the gene hly coding for the hemolysin of Listeria monocytogenes is under the control of the promoter Ptet.

In a preferred embodiment, the vectorial system according to the invention is characterized in that said E. coli strain was made incapable of surviving in said cells as soon as it enters the cytoplasm of eukaryotic cells.

The E. coli strain may be made incapable of multiplying and of surviving in eukaryotic cells in multiple ways, in particular by making it auxotrophic for a factor necessary for its survival and absent from the eukaryotic cells.

In the preferred embodiment according to the invention, the E. coli bacterium is made incapable of multiplying and therefore of surviving as soon as it enters the cytoplasm of eukaryotic cells. To do this, in a preferred embodiment according to the invention, said E. coli bacterium was modified so as to be made auxotrophic for diaminopimelic acid which is an essential compound of the synthesis of the bacterial wall and the biosynthesis route of which is well known.

In a still preferred embodiment, the E. coli bacterium is made dap⁻ following a double event of homologous recombination in two genes for synthesis of diaminopimelic acid which is specific of prokaryotes and absent in the cytoplasm of eukaryotic cells.

The first two steps of the synthesis of diaminopimelate which is not present in the cells of mammals, are catalyzed by the enzymes coded by the genes dapA and dapB.

The gene may be doubly inactivated by deletion and insertion-inactivation in the genes in dapA and dapB.

The inactivation of the metabolic chain at the first step avoids the accumulation in the cells of a metabolic intermediate which may be metabolized by another enzyme of the cell.

In a preferred embodiment, the vectorial system according to the invention is characterized in that one of the genes imparting to said non-pathogenic recombinant E. coli bacterium, the capacity of penetrating into the cytoplasm of said eukaryotic target cells is integrated into the chromosome of said E. coli by homologous recombination at the gene dapA or dapB of said non-pathogenic E. coli bacterium, resulting in an at least partial deletion of this gene and in its inactivation.

In a preferred embodiment, the vectorial system according to the invention is characterized in that one of the genes or both genes imparting to said non-pathogenic recombinant E. coli bacterium, the capacity of penetrating into the cytoplasm of said eukaryotic targeted cells, are integrated into the chromosome of said E. coli by:
insertion of the penetration gene and deletion of the dapA gene between its 5′ fragment of sequence SEQ ID NO: 3 and its 3′ fragment of sequence SEQ ID NO: 5, or between a 5′ and 3′ fragment of the dapA gene resulting in the deletion of a fragment of the dapA gene, sufficient for inactivating this gene and/or making auxotrophic said non-pathogenic E. coli bacterium for diaminopimelic acid;
insertion of the lysis gene and deletion of the dapB gene between its 5′ fragment of sequence SEQ ID NO: 9 and its 3′ fragment of sequence SEQ ID NO: 11, or between a 5′ and 3′ fragment of the dapB gene resulting in the deletion of a fragment of the dapB gene, sufficient for inactivating this gene and/or making auxotrophic said non-pathogenic E. coli bacterium for diaminopimelic acid.

In a preferred embodiment, the vectorial system according to the invention is characterized in that the selection of the non-pathogenic recombinant E. coli bacteria having integrated the penetration gene, is made by means of a tetracyclin resistance cassette flanked with FRT sites allowing its excision from the bacterial chromosome by the yeast Flp recombinase, preferably by introducing a plasmid in said E. coli bacterium capable of producing the Flp recombinase in transient form.

In a preferred embodiment, the vectorial system according to the invention is characterized in that the chromosomal integration of the gene(s) imparting to said non-pathogenic recombinant E. coli bacterium the capacity of penetrating into the cytoplasm of said eukaryotic target cells, is carried out by homologous recombination in the presence of Rcd α(exo) and Rcd β(bet) proteins of the bacteriophage λ expressed by a plasmid.

In a preferred embodiment, the vectorial system according to the invention is characterized in that this is a non-pathogenic recombinant E. coli bacterium of genotype:
[thi-1, endA1, hsdR17 (rκ⁻ mκ⁺), supE44, Δ(lac)X74, ΔdapAΩinv, AdapBΩhly, recA1]; or
[BM2710, ΔdapAΩinv, AdapBΩhly] or [BM2710, ΔdapAΩPtrc-inv, AdapBΩPtet-hly], the strain BM2710 having been deposited on Oct. 27, 1995 under number I-1635 at the CNCM; or
the strain E. coli BM4570 deposited on Jul. 18, 2007 under number I-3788 at the CNCM (Collection Nationale de Cultures de Microorganismes), Institut Pasteur, 25 Rue du Docteur Roux, 75724 Paris Cédex 15, France; or
the strain E. coli BM4569 deposited on Dec. 13, 2007 under number I-3877 at the CNCM (Collection Nationale de Cultures de Microorganismes), Institut Pasteur, 25 Rue du Docteur Roux, 75724 Paris Cedex 15, France; or
the strain E. coli BM4658 deposited on Jan. 17, 2008 under number I-3894 at the CNCM (Collection Nationale de Cultures de Microorganismes), Institut Pasteur, 25 Rue du Docteur Roux, 75724 Paris Cédex 15, France.

One of the problems during the in vivo use of E. coli is its potential toxicity related to LPS. It was shown recently that the pro-inflammatory potential of LPS may be reduced by attenuating the immunogenicity of the lipid A by a genetic modification (Somerville et al., 1996, d'Hauteville et al., 2002).

Thus, under a particular aspect, the object of the present invention is a vectorial system according to the invention, characterized in that the gene of structure msbB of said non-pathogenic recombinant E. coli bacterium was mutated in order to generate a strain for which the lipid A of the LPS is free of any myristoyl fatty acids.

In a particularly preferred embodiment the vectorial system according to the invention is characterized in that this is a non-pathogenic recombinant E. coli bacterium of genotype:
[thi-1, endA1, hsdR17 (rκ⁻ mκ⁺), supE44, Δ(lac)X74, ΔmsbB, ΔdapAΩinv, AdapBΩhly, recA1]; or
[BM2710, ΔmsbB, ΔdapAΩinv, AdapBΩhly] or [BM2710, ΔmsbB, ΔdapAΩPtrc-inv, AdapBΩPtet-hly], the strain BM2710 having been deposited on Oct. 27, 1995 under number I-1635 at the CNCM; or
the strain E. coli BM4573 deposited on Jul. 18, 2007 under number I-3790 at the CNCM (Collection Nationale de Cultures de Microorganismes), Institut Pasteur, 25 Rue du Docteur Roux, 75724 Paris Cédex 15, France; or
the strain E. coli BM4571 deposited on Dec. 13, 2007 under number I-3878 at the CNCM (Collection Nationale de Cultures de Microorganismes), Institut Pasteur, 25 Rue du Docteur Roux, 75724 Paris Cedex 15, France; or
the strain E. coli BM4572 deposited on Dec. 13, 2007 under number I-3879 at the CNCM (Collection Nationale de Cultures de Microorganismes), Institut Pasteur, 25 Rue du Docteur Roux, 75724 Paris Cedex 15, France; or
the strain E. coli BM4657 deposited on Jan. 17, 2008 under number I-3893 at the CNCM (Collection Nationale de Cultures de Microorganismes), Institut Pasteur, 25 Rue du Docteur Roux, 75724 Paris Cédex 15, France.

The present invention also allows the generation of an invasive E. coli strain allowing the production in a large amount by the vector, of heterologous proteins and of short hairpin RNA (sh RNA, interfering RNA molecules synthesized by the bacterium) under the control of the promoter T7, the gene coding for T7 RNA polymerase is integrated into the bacterial chromosome.

Thus, the present invention comprises an in vitro, ex vivo or in vivo method for producing heterologous proteins or a nucleic acid, notably RNAs of the short hairpin RNA type, in an eukaryotic cell, characterized in that it applies a vectorial system according to the invention wherein said nucleic acid is under the control of the promoter T7 and the gene coding for T7 RNA polymerase is integrated into the bacterial chromosome of said recombinant E. coli bacterium.

In a preferred embodiment the vectorial system according to the invention is characterized in that the gene of the T7 RNA polymerase has been integrated, preferably under the control of the promoter lacUV5, into the chromosome of said non-pathogenic recombinant E. coli bacterium.

In a particularly preferred embodiment, the vectorial system according to the invention is characterized in that this is a non-pathogenic recombinant E. coli bacterium of genotype:
[thi-1, endA1, hsdR17 (rκ mκ⁻), supE44, Δ(lac)X74, ΔmsbB, ΔdapAΩinv, AdapBΩhly, recA1, (DE3)]; or
[BM2710, ΔdapAΩinv, AdapBΩhly, (DE3)] or [BM2710, ΔdapAΩPtrc-inv, AdapBΩPtet-hly, (DE3)], the strain BM2710 having been deposited on Oct. 27, 1995 under number I-1635 at the CNCM; or
the strain E. coli BM4570 (DE3) deposited on Jul. 18, 2007 under number I-3789 at the CNCM (Collection Nationale de Cultures de Microorganismes), Institut Pasteur, 25 Rue du Docteur Roux, 75724 Paris Cédex 15, France.

In a preferred embodiment, the vectorial system according to the invention is characterized in that said non-pathogenic recombinant E. coli strain is transformed by a vector either replicative or not, in E. coli bearing said nucleic acid of interest or coding for said protein of interest, and if necessary, placed under the control of regulation elements in said eukaryotic target cells.

Advantageously, said nucleic acid of interest includes a DNA fragment coding for a protein of interest, the latter fragment being placed under the control of regulation elements in said eukaryotic target cells.

By “regulation elements” are meant suitable regulating sequences for transcription and then translation such as a promoter, including “start” and “stop”, “enhancer” and “operator” codons. The means and methods for identifying and selecting these elements are well known to one skilled in the art.

In an advantageous embodiment, said strain is transformed by a replicative or non-replicative vector in E. coli bearing said nucleic acid of interest and optionally said regulation elements.

In a preferred embodiment, the vectorial system according to the invention is characterized in that said vector bearing said nucleic acid of interest further includes elements for integration into the genome of target eukaryotic cells.

In a preferred embodiment, the vectorial system according to the invention is characterized in that said vector bearing said nucleic acid of interest further includes a replication origin allowing the vector to replicate extrachromosomally in said target eukaryotic cells.

According to the present invention, said nucleic acid of interest is borne by a replicative and non-integrative plasmid in the bacterium. Further, the foreign DNA fragment is placed under the control of functional expression elements in the target eukaryotic cells.

Said carrier vector may be replicative or integrative in the target eukaryotic cells, i.e. it may include elements for integration into the genome of the target eukaryotic cells, or include a replication origin allowing the vector to replicate extrachromosomally in the target eukaryotic cells.

In a preferred embodiment, the vectorial system according to the invention is characterized in that said eukaryotic cells are mammalian cells, yeast or plant cells, preferably mammalian cells.

Under another aspect, the object of the present invention is a vectorial system according to the invention for preparing a therapeutic composition.

Under another aspect, the object of the present invention is a method for in vivo or in vitro transfer of DNA into eukaryotic cells other than animal or human cells, characterized in that it applies a vectorial system according to the invention.

Under another aspect, the object of the present invention is an in vivo or ex vivo method for transferring DNA into human or animal eukaryotic cells from a biological sample of human or animal origin, characterized in that it applies a vectorial system according to the invention.

Under another aspect, the object of the present invention is the use of a vectorial system according to the invention, for preparing a vaccinal composition, characterized in that the nucleic acid of interest codes for an antigen of an infectious agent, the infection of which may be prevented by means of an antibody directed against this antigen.

Under another aspect, the object of the present invention is the use of a vectorial system according to the invention, for the preparation of an anti-tumoral vaccinal composition, characterized in that the nucleic acid of interest codes for a tumoral antigen, the tumor or cancer of which may be prevented by means of an antibody directed against this antigen.

Under a last aspect, the object of the present invention is the use of a vectorial system according to the invention, for preparing a therapeutic composition intended for treating or preventing diseases by gene therapy.

Other features and advantages of the present invention will become apparent in the light of the examples and of the figures hereafter.

### CAPTIONS OF THE FIGURES

FIG. 1: FIG. 1 illustrates a diagram for constructing bacterial vectors.

FIG. 2: FIG. 2 illustrates a diagram showing the different steps of constructions in order to end up with the different vectorial systems BM4570, BM4573 and BM4570 (DE3).

FIGS. 3A-3C: FIGS. 3A-3C illustrate comparative results obtained by FACS analysis for the different vectorial systems derived from the strain BM2710, expression of the inv gene at the surface of the E. coli bacterium.

FIG. 4: FIG. 4 illustrates the quantification of the hemolytic activity in the three vectors.

FIG. 5: FIG. 5 illustrates the capacity of transferring the gene pEGFP-C1 by BM4570 by comparison with that of BM2710pGB2Ωinv-hly.

### EXAMPLE 1

### Construction of E. coli BM4570 by Integration of the Inv and Hly Genes into the Chromosome of E. coli BM2710

It was shown that the expression of the Red α(exo) and Red β(bet) proteins of the bacteriophage λ, as well as of the Red γ (gam) protein was able to efficiently promote homologous recombination in an E. coli recA strain without any homologous recombination. By using this system, it is therefore possible to carry out allele exchange of genes located in the chromosome following a dual recombination event by electroporation in the strain to be modified of a DNA fragment containing terminal sequences homologous to the target (J. P. P. Muyrers, 2001). The Red αβγ functions of the phage λ are borne by the plasmid pKOBEGA which has a heat-sensitive replication origin and are expressed under the control of the inducible promoter by arabinose pBAD (Chaveroche et al., 200).

The E. coli strain harboring pKOBEGA was electro-transformed with fragments which include the gene inv or the gene hly respectively flanked by the 5′ and 3′ portions of the gene dapA or of the gene dapB.

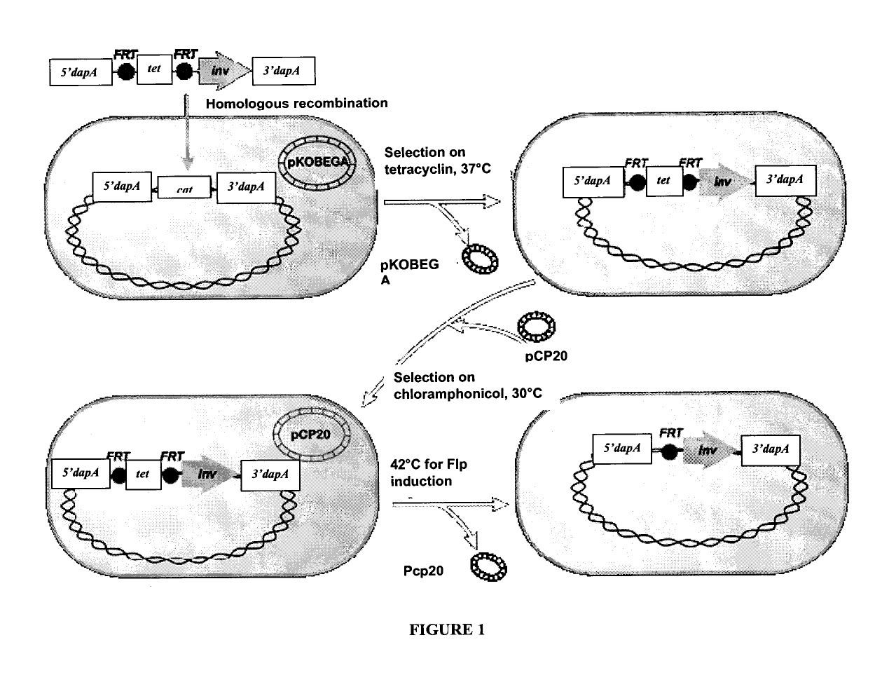

In order to select the recombinant strains, we used a tetracyclin resistance cassette which it has been possible to secondarily excise from the chromosome. The cassette tet^{R} (D. Mazel, unpublished) is flanked by short direct repetitions (FRT sites) which allow its excision by the Flp recombinase of yeast (M. M. Cox, 1983) thereby generating a stable bacterial vector and without any resistance character. Resistance to tetracyclin was deleted from the chromosome by introducing into the strain, the plasmid pCP20 (Cherepanov and Wackermagel, 1995) which allows transient production of the Flp recombinase in trans (FIG. 1).

We have integrated with this system:
into the gene dapA of E. coli BM2710, the inv gene under the control of the strong promoter Ptrc. Ptrc is a hybrid promoter consisting of the sequence −35 of the promoter trp and of the sequence −10 of lacUV5;
in the gene dapB of E. coli BM2710, the gene hly without any signal sequence under the control of the promoter Ptet of pACYC184 (Higgins et al., 1999).

### EXAMPLE 2

### Construction of E. coli BM4573 from Invasive E. coli BM2710 with Modified LPS

Mutations in the structure gene msbB of E. coli generating strains in the lipid A of the LPS is free of myristoyl fatty acids which thus reduces their pro-inflammatory activity (Somerville et al., 1996).

The plasmid pCVD442 msbB1::km, a suicide vector which only replicates in strains producing the protein Pir, was used (d'Hauteville et al., 2002). The insertion in this plasmid comprises the 5′ portion and the region upstream from the mspB gene, the determinant of resistance to kanamycin aphA of pUC4K and the 3′ portion and the region downstream from the msbB gene. The gene aphA of this construction was replaced with the tet^{R} cassette flanked with FRT sites. The strain of E. coli BM4573 (ΔmsbB) was constructed by using the same approach as the one described earlier.

### EXAMPLE 3

### Construction of E. coli BM4570 (DE3) by Integration of the Gene of T7 RNA Polymerase into the Chromosome of E. coli BM4570

The λDE3 lysogenization kit (Novagen) was used for integrating the gene of T7 RNA polymerase under the control of the promoter lacUV5.

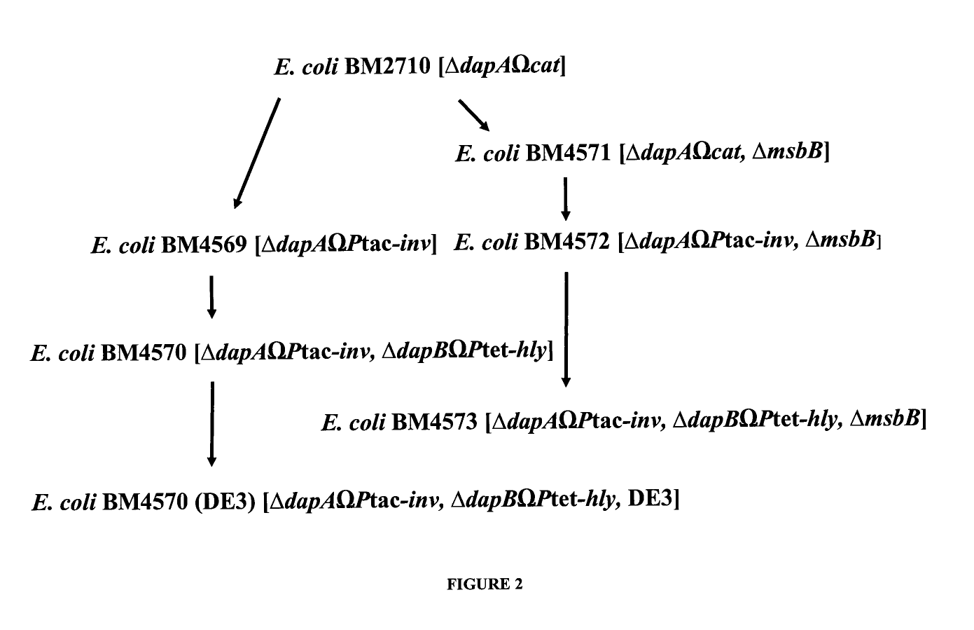

The main steps of the construction of the strains are illustrated in FIG. 2.

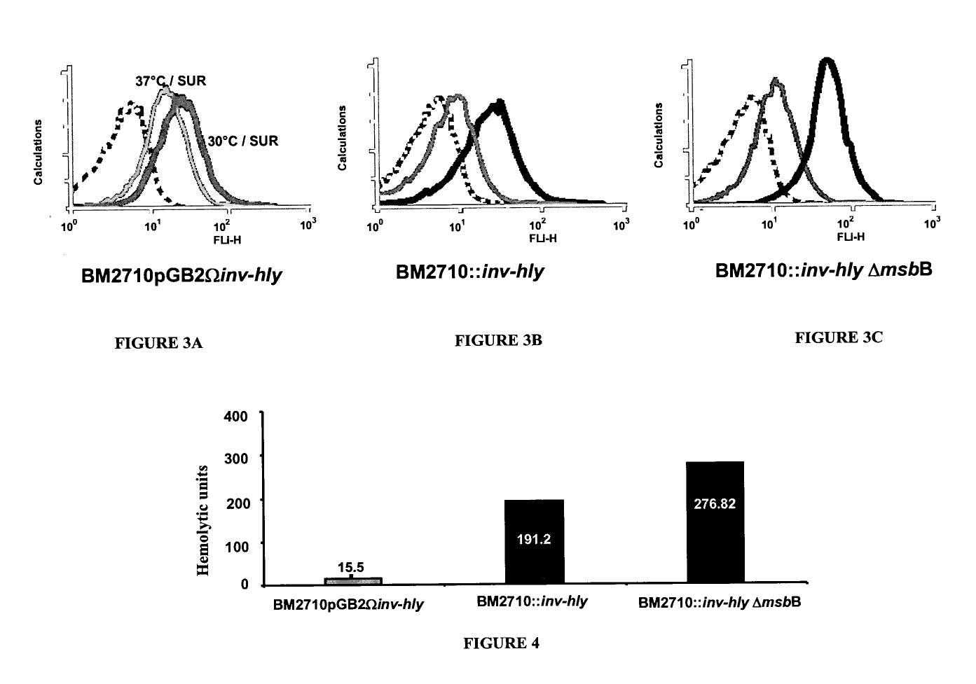

For each of the constructed strains, the expression of invasin at the surface of the bacterium and the production of listeriolysin were studied and compared with those of the original strain which harbors the pGB2Ωinv-hly plasmid (FIG. 3).

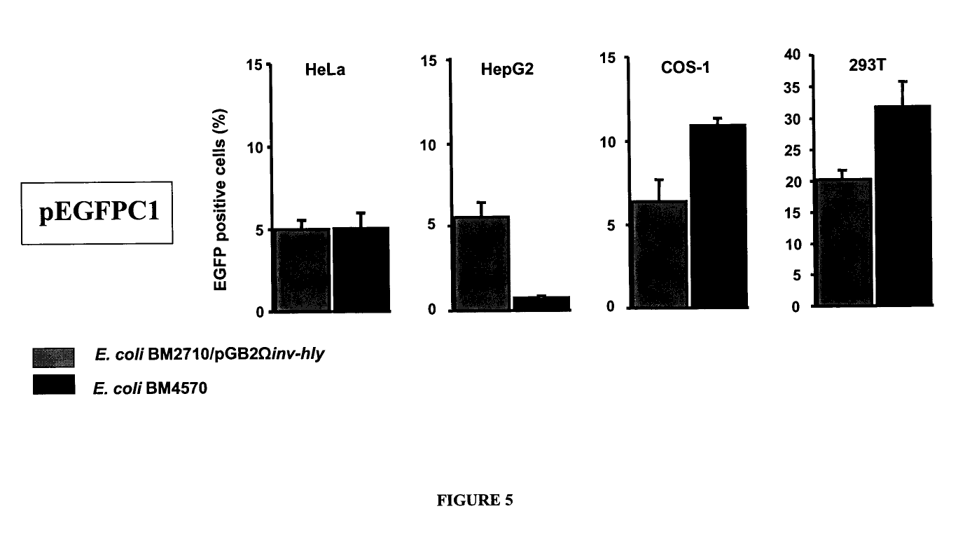

Also, the capacity of these strains of transferring the EGFP gene was studied comparatively (FIG. 5).

### BIBLIOGRAPHIC REFERENCES

Chaveroche M K., Ghigo J M, and d'Enfert C (2000) A rapid method for efficient gene replacement in the filamentous fungus Aspergillus nidulans. Nucleic Acids Res., 28 (22) e97.
Courvalin P, Goussard S and Grillot-Courvalin C (1995) Gene transfer from bacteria to mammalian cells. C. R. Acad. Sci. Ser. III., 318:1207-1212.
Cox M M. (1983) The FLP protein of the yeast 2-μm plasmid: Expression of a eukaryotic genetic recombination system in Escherichia coli. Proc. Natl. Acad. Sci. USA, 80:4223-4227.
d'Hauteville H, Khan S, Maskell D J, Kussak A, Weintraub A, Mathison J, Ulevitch R J, Wuscher N, Parsot C, and Sansonetti P J (2002) Two msbB genes encoding maximal acylation of lipid A are required for invasive Shigella flexneri to mediate inflammatory rupture and destruction of the intestinal epithelium. J. Immunol., 168:5240-5251.
Grillot-Courvalin C, Goussard S, Huetz F, Ojeius D M, and Courvalin P (1998) Functional gene transfer from intracellular bacteria to mammalian cells. Nature Biotechnol., 16:862-866.
Robbens J, Racymackers A, Steidlcr L, Fiers W, and Remaut E (1995) Production of soluble and active recombinant murine interleukin-2 in Escherichia coli: high level expression, Kil-induced release, and purification. Protein Expr. Purif., 6:481-486.
Somerville Jr. J E, Cassiano L, Bainbridge B, Cunningham M D, and Darveau R P (1996) A novel Escherichia coli lipid A mutant that produces an antiinflammatory lipopolysaccharide. J. Clin. Invest., 97 (2):359-365.

1. A vectorial system capable of delivering in eukaryotic target cells a nucleic acid of interest, or coding for a protein of interest, comprising a non-pathogenic recombinant bacterium E. coli, said non-pathogenic bacterium E. coli being further:
modified by introduction (by allele exchange) of one or more genes imparting to this non-pathogenic recombinant E. coli bacterium the capacity of penetrating into the cytoplasm of said eukaryotic target cells;
incapable of surviving in the cytoplasm of said target cell; and
modified by foreign DNA fragments,
characterized in that the gene(s) imparting to said non-pathogenic recombinant E. coli bacterium, the capacity of penetrating into the cytoplasm of said eukaryotic target cells are integrated into the chromosome of said non-pathogenic E. coli.

2. The vectorial system according to claim 1, characterized in that said non-pathogenic bacterium E. coli is transformed by chromosomal integration of two genes, stemming from one or two other bacteria, imparting to it the capacity of penetrating into the cytoplasm of said target cells.

3. The vectorial system according to any of claim 1 or 2, characterized in that the gene(s) integrated into the chromosome of the non-pathogenic bacterium E. coli and imparting to it its capacity of penetrating into the cytoplasm of said target cells, allow it to lyze the membrane of the vacuoles of said target cells in order to reach the cytoplasm.

4. The vectorial system according to any of claims 1 to 3, characterized in that said non-pathogenic bacterium E. coli is transformed by chromosomal integration by the invasion gene of the bacterium Y. pseudotuberculosis which imparts the property of penetrating into the cytoplasm of epithelial cells.

5. The vectorial system according to any of claims 1 to 3, characterized in that said non-pathogenic bacterium E. coli is transformed by chromosomal integration by the gene coding for the hemolysin of L. monocytogenes which imparts the property of lyzing the membrane of the vacuoles of said target cells.

6. The vectorial system according to any of claims 1 to 5, characterized in that said non-pathogenic bacterium E. coli is transformed by chromosomal integration both by the invasion gene of the bacterium Y. pseudotuberculosis which imparts the property of penetrating into the cytoplasm of epithelial cells and by the gene coding for the hemolysin of L. monocytogenes which imparts the property of lyzing the membrane of the vacuoles of said target cells and by the gene.

7. The vectorial system according to any of claims 1 to 6, characterized in that said non-pathogenic bacterium E. coli is transformed by chromosomal integration both by:
1. the invasion gene inv of Yersinia pseudotuberculosis, preferably of sequence SEQ ID NO: 10 or of a sequence identical to at least 80% thereof and capable of imparting the property of penetrating into the cytoplasm of epithelial cells; and
2. the gene hly coding for the hemolysin of Listeria monocytogenes, preferably of sequence SEQ ID NO: 4 or of a sequence identical to at least 80% thereof, and capable of imparting the property of lyzing the membranes of the vacuoles.

8. The vectorial system according to claim 7, characterized in that:
1. the invasion gene inv of Yersinia pseudotuberculosis is under the control of the promoter Ptet;
2. the gene hly coding for the hemolysin of Listeria monocytogenes is under the control of the promoter Ptrc.

9. The vectorial system according to any of claims 1 to 8, characterized in that said E. coli strain was made incapable of surviving in said cells as soon as it enters the cytoplasm of eukaryotic cells.

10. The vectorial system according to claim 9, characterized in that said non-pathogenic recombinant E. coli bacterium was modified so as to make it auxotrophic for diaminopimelic acid.

11. The vectorial system according to claim 10, characterized in that the gene imparting to said non-pathogenic recombinant E. coli bacterium, the capacity of penetrating into the cytoplasm of said eukaryotic target cells is integrated into the chromosome of said E. coli by homologous recombination at the gene dapA of said non-pathogenic E. coli bacterium, resulting in an at least partial deletion of this gene and in its inactivation, preferably that the gene dapA coding for the enzyme dihydropicolinate synthase is inactivated.

12. The vectorial system according to claim 10 or 11, characterized in that the gene imparting to said non-pathogenic recombinant E. coli bacterium, the capacity of penetrating into the cytoplasm of said eukaryotic target cells is integrated into the chromosome of said E. coli bacterium by homologous recombination at the gene dapB of said non-pathogenic E. coli bacterium, resulting in an at least partial deletion of this gene and in its inactivation.

13. The vectorial system according to any of claims 9 to 12, characterized in that the gene imparting to said non-pathogenic recombinant E. coli bacterium, the capacity of penetrating into the cytoplasm of said eukaryotic target cells and of lyzing the penetration vacuole is integrated into the chromosome of said E. coli bacterium by:
insertion of the penetration gene and deletion of the dapA gene between its 5′ fragment of sequence SEQ ID NO: 3 and its 3′ fragment of sequence SEQ ID NO: 5, or between a 5′ and 3′ fragment of the dapA gene resulting in the deletion of a fragment of the dapA gene, sufficient for inactivating this gene and/or making auxotrophic said non-pathogenic E. coli bacterium for diaminopimelic acid;
insertion of the lysis gene and deletion of the dapB gene between its 5′ fragment of sequence SEQ ID NO: 9 and its 3′ fragment of sequence SEQ ID NO: 11, or between a 5′ and 3′ fragment of the dapB gene resulting in the deletion of a fragment of the dapB gene, sufficient for inactivating this gene and/or making auxotrophic said non-pathogenic E. coli bacterium for diaminopimelic acid.

14. The vectorial system according to any of claims 9 to 13, characterized in that the selection of the non-pathogenic recombinant E. coli bacteria having integrated the penetration gene and of the lysis gene, is made by means of a tetracyclin resistance cassette flanked with FRT sites allowing its excision from the bacterial chromosome by the yeast Flp recombinase, preferably by introducing a plasmid in said E. coli bacterium capable of producing the Flp recombinase in a transient form.

15. The vectorial system according to any of claims 1 to 14, characterized in that the chromosomal integration of the gene(s) imparting to said non-pathogenic recombinant E. coli bacterium the capacity of penetrating into the cytoplasm and of lyzing the penetration vacuole of said eukaryotic target cells, is achieved by homologous recombination in the presence of Red α(exo) and Red β(bet) proteins of the bacteriophage λ, expressed by a plasmid.

16. The vectorial system according to any of claims 1 to 15, characterized in that this is a non-pathogenic recombinant E. coli bacterium of genotype:
[thi-1, endA1, hsdR17 (rκ⁻ mκ⁺), supE44, Δ(lac)X74, ΔdapAΩinv, AdapBΩhly, recA1]; or
[BM2710, ΔdapAΩinv, AdapBΩhly] or [BM2710, ΔdapAΩPtrc-inv, AdapBΩPtet-hly], the strain BM2710 having been deposited on Oct. 27, 1995 under number I-1635 at the CNCM; or
the strain E. coli BM4570 deposited on Jul. 18, 2007 under number I-3788 at the CNCM (Collection Nationale de Cultures de Microorganismes), Institut Pasteur, 25 Rue du Docteur Roux, 75724 Paris Cédex 15, France; or
the strain E. coli BM4569 deposited on Dec. 13, 2007 under number I-3877 at the CNCM (Collection Nationale de Cultures de Microorganismes), Institut Pasteur, 25 Rue du Docteur Roux, 75724 Paris Cédex 15, France; or
the strain E. coli BM4658 deposited on Jan. 17, 2008 under number I-3894 at the CNCM (Collection Nationale de Cultures de Microorganismes), Institut Pasteur, 25 Rue du Docteur Roux, 75724 Paris Cédex 15, France.

17. The vectorial system according to any of claims 1 to 16, characterized in that the gene of structure msbB of said non-pathogenic recombinant E. coli bacterium was mutated in order to generate a strain for which the lipid A of the LPS is without any myristoyl fatty acids.

18. The vectorial system according to claim 17, characterized in that this is a non-pathogenic recombinant E. coli bacterium of genotype:
[thi-1, endA1, hsdR17 (rκ⁻ mκ⁺), supE44, Δ(lac)X74, ΔmsbB, ΔdapAΩinv, AdapBΩhly, recA1]; or
[BM2710, ΔmsbB, ΔdapAΩinv, AdapBΩhly] or [BM2710, ΔmsbB, ΔdapAΩPtrc-inv, AdapBΩPtet-hly], the strain BM2710 having been deposited on Oct. 27, 1995 under number I-1635 at the CNCM of genotype [thi-1, endA1, hsdR17 (rκ⁻ mκ⁺), supE44, Δ(lac)X74, ΔdapAΩ, recA1]; or
the strain E. coli BM4573 deposited on Jul. 18, 2007 under number I-3790 at the CNCM (Collection Nationale de Cultures de Microorganismes), Institut Pasteur, 25 Rue du Docteur Roux, 75724 Paris Cédex 15, France; or
the strain E. coli BM4571 deposited on Dec. 13, 2007 under number I-3878 at the CNCM (Collection Nationale de Cultures de Microorganismes), Institut Pasteur, 25 Rue du Docteur Roux, 75724 Paris Cedex 15, France; or
the strain E. coli BM4572 deposited on Dec. 13, 2007 under number I-3879 at the CNCM (Collection Nationale de Cultures de Microorganismes), Institut Pasteur, 25 Rue du Docteur Roux, 75724 Paris Cédex 15, France; or
the strain E. coli BM4657 deposited on Jan. 17, 2008 under number I-3893 at the CNCM (Collection Nationale de Cultures de Microorganismes), Institut Pasteur, 25 Rue du Docteur Roux, 75724 Paris Cédex 15, France.

19. The vectorial system according to any of claims 1 to 18, characterized in that said nucleic acid of interest is of all sizes up to 100 kb or from 100 to 150 kb or greater than 150 kb.

20. The vectorial system according to any of claims 1 to 18, characterized in that said nucleic acid of interest, preferably coding for a heterologous protein or a RNA of the shRNA type, is under the control of the promoter T7, the gene coding for the T7 RNA polymerase being integrated into the bacterial chromosome.

21. The vectorial system according to any of claims 1 to 18, characterized in that the gene of the T7 RNA polymerase has been integrated, preferably under the control of the promoter lacUV5, into the chromosome of said non-pathogenic recombinant E. coli bacterium.

22. The vectorial system according to claim 21, characterized in that this is a non-pathogenic recombinant E. coli bacterium of genotype:
[thi-1, endA1, hsdR17 (rκ⁻ mκ⁻), supE44, Δ(lac)X74, ΔmsbB, ΔdapAΩinv, AdapBΩhly, recA1, (DE3)]; or
[BM2710, ΔdapAΩinv, AdapBΩhly, (DE3)] or [BM2710, ΔdapAΩPtrc-inv, AdapBΩPtet-hly, (DE3)], the strain BM2710 having been deposited on Oct. 27, 1995 under number I-1635 at the CNCM of genotype [thi-1, endA1, hsdR17 (rκ⁻ mκ⁻), supE44, Δ(lac)X74, ΔdapAΩ, recA1]; or
the strain E. coli BM4570 (DE3) deposited on Jul. 18, 2007 under number I-3789 at the CNCM (Collection Nationale de Cultures de Microorganismes), Institut Pasteur, 25 Rue du Docteur Roux, 75724 Paris Cédex 15, France.

23. The vectorial system according to any of claims 1 to 22, characterized in that said non-pathogenic recombinant E. coli strain is transformed by a vector either replicative or not, in E. coli bearing said nucleic acid of interest or coding for said protein of interest, and if necessary, placed under the control of regulation elements in said eukaryotic target cells.

24. The vectorial system according to claim 22 or 23, characterized in that said vector bearing said nucleic acid of interest further includes elements for integration into the genome of target eukaryotic cells.

25. The vectorial system according to claim 22 or 23, characterized in that said vector bearing said nucleic acid of interest further includes a replication origin allowing the vector to replicate extrachromosomally in said eukaryotic target cells.

26. The vectorial system according to any of claims 1 to 25, characterized in that said eukaryotic cells are mammalian cells, yeast or plant cells, preferably mammalian cells.

27. The vectorial system according to any of claims 1 to 26, for the preparation of a therapeutic composition.

28. A method for in vivo or in vitro transfer of DNA in eukaryotic cells other than animal or human cells, characterized in that it applies a vectorial system according to any of claims 1 to 26.

29. A method for in vitro or ex vivo transfer of DNA and RNA in human or animal eukaryotic cells from a biological sample of human or animal origin, characterized in that it applies a vectorial system according to any of claims 1 to 26.

30. The use of a vectorial system according to any of claims 1 to 27 for preparing a vaccinal composition characterized in that the nucleic acid of interest codes for one or more antigens of an infectious agent or for antigen fragments.

31. The use of a vectorial system according to any of claims 1 to 27 for preparing an anti-tumoral vaccinal composition characterized in that the nucleic acid of interest codes for an anti-tumoral antigen.

32. The use of a vectorial system according to any of claims 1 to 27 for preparing a therapeutic composition intended for treating and preventing diseases by gene therapy.


SEQUENCE LISTING (12 sequences)

SEQ ID NO: 1 - DNA, 4398 bp, Artificial sequence
  Delta dapB omega hly sequence from BM4570
cgctcgcccaaggggccgctgtgccactggtacgcgcaccgcgtgatgcggcatctgtgcacggcgaatcgggaatggctggctacgactttgttgagcacaaccgaaagccgctcgggataccggcgtttctggcgattcgggatgccctgatgcgtgcaccagagcctgttaccctggtggccatcggcccgttaaccaatattgcgctgttactttcacaatgcccggaatgcaagccgtatattcgccgtctggtgatcatgggtggttctgccggacgcggcaactgtacgccaaacgccgagtttaatattgctgccgatccagaagctgctgcctgtgtcttccgcagtggtattgaaatcgtcatgtgcggtttggatgtcaccaatcaggcaatattaactcctgactatctctctacactgccgcagttaaaccgtaccgggaaaatgcttcacgccctgtttagccactaccgtagcggcagtatgcaaagcggcttgcgaatgcacgatctctgcgccatcgcctggctggtgcgcccggacctgttcactctcaaaccctgttttgtggcagtggaaactcagggcgaatttacctcaggcacgacggtggttgatatcgacggttgcctgggcaagccagccaatgtacaggtggcattggatctggatgtgaaaggcttccagcagtgggtggctgaggtgctggctctggcgtcgtaacctgtcacatgttattggcatgcagtcattcatcgactcatgcctttcactgatatccctccctgtttatcattaatttctaattatcagcgtttttggctggcggcgtagcgatgcgctggttactctgaaaacggtctatgcaaattaacaaaagagaatagctatgcatgatgcaaacatccgcgttgccatcgcgggagccggggggcgtatgggccgccagttgattcaggcggcgctggcattagagggcgtgcagttgggcgctgcgctggagcgtgaaggatcttctttactgggcagcgacgccggtgagctggccggagccgggaaaacaggcgttaccgtgcaaagcagcctcgatgcggtaaaagatgattttgatgtgtttatcgattttacccgtccggaaggtacgctgaaccatctcgctttttgtcgccagcatggcaaagggatggtgatcggcactacggggtttgacgaagccggtaaacaagcaattcgtgacgccgctgccgatattgcgattgtctttgctgccaattttagcgttggcgttaacgtcatgcttaagctgctggagaaagcagccaaagtgatgggtgactacactagataactgaggatctgaagttcctatactttctagagaataggaacttcggaataggaacttcagatcctcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcctctagaaataattttgtttaactttaagaaggagatatacatatgaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgcctggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaataataactgcagctgaaagattgcgcggtctacagtcgtgaaggccacaccggtgaacgtgtgcctggcaccattggttttgccaccgtgcgtgcaggtgacatcgttggtgaacataccgcgatgtttgccgatattggcgagcgtctggagatcacccataaggcgtccagccgtatgacatttgctaacggcgcggtaagatcggctttgtggttgagtggtaaggaaagcggtctttttgatatgcgagatgtacttgatctcaataatttgtaaccacaaaatatttgttatggtgcaaaaataacacatttaatttattgattataaagggctttaatttttggcccttttatttttggtgttatgtttttaaattgtctataagtgccaaaaattacatgttttgtcttctgtttttgttgttttaatgtaaattttgaccatttggtccacttttttctgctcgtttttatttcatgcaatcttcttgctgcgcaagcgttttccagaacaggttagatgatctttttgtcgcttaatgcctgtaaaacatgcatgagccacaaaataatataaaaaatcccgccattaagttgacttttagcgcccatatctccagaatgccgccgtttgccagaaattcgtcggtaagcagatttgcattgatttacgtcatcattgtgaattaatatgcaaataaagtgagtgaatattctctggagggtgttttgattaagtcagcgctattggttctggaagacggaacccagtttcacggtcgggccataggggcaacaggttcggcggttggggaagtcgttttcaatacttcaatgaccggttatcaagaaatcctcactgatccttcctattctcgtcaaatcgttactcttacttatccccatattggcaatgtcggcaccaatgacgccgatgaagaatcttctcaggtacatgcacaaggtctggtgattcgcgacctgccgctgattgccagcaacttccgtaataccgaagacctctcttcttacctgaaacgccataacatcgtggcgattgccgatatcgatacccgtaagctgacgcgtttactgcgcgagaaaggcgcacagaatggctgcattatcgcgggcgataacccggatgcggcgctggcgttagaaaaagcccgcg

SEQ ID NO: 2 - DNA, 975 bp, Artificial sequence
  dapB 5′ flanking sequence
cgctcgcccaaggggccgctgtgccactggtacgcgcaccgcgtgatgcggcatctgtgcacggcgaatcgggaatggctggctacgactttgttgagcacaaccgaaagccgctcgggataccggcgtttctggcgattcgggatgccctgatgcgtgcaccagagcctgttaccctggtggccatcggcccgttaaccaatattgcgctgttactttcacaatgcccggaatgcaagccgtatattcgccgtctggtgatcatgggtggttctgccggacgcggcaactgtacgccaaacgccgagtttaatattgctgccgatccagaagctgctgcctgtgtcttccgcagtggtattgaaatcgtcatgtgcggtttggatgtcaccaatcaggcaatattaactcctgactatctctctacactgccgcagttaaaccgtaccgggaaaatgcttcacgccctgtttagccactaccgtagcggcagtatgcaaagcggcttgcgaatgcacgatctctgcgccatcgcctggctggtgcgcccggacctgttcactctcaaaccctgttttgtggcagtggaaactcagggcgaatttacctcaggcacgacggtggttgatatcgacggttgcctgggcaagccagccaatgtacaggtggcattggatctggatgtgaaaggcttccagcagtgggtggctgaggtgctggctctggcgtcgtaacctgtcacatgttattggcatgcagtcattcatcgactcatgcctttcactgatatccctccctgtttatcattaatttctaattatcagcgtttttggctggcggcgtagcgatgcgctggttactctgaaaacggtctatgcaaattaacaaaagagaatagctatgcatgatgcaaacatccgcgttgccatcgcgggagccggggggcgtatgggccgccagttgattcaggcggcg

SEQ ID NO: 3 - DNA, 377 bp, Artificial sequence
  dapB 5′ sequence
ctggcattagagggcgtgcagttgggcgctgcgctggagcgtgaaggatcttctttactgggcagcgacgccggtgagctggccggagccgggaaaacaggcgttaccgtgcaaagcagcctcgatgcggtaaaagatgattttgatgtgtttatcgattttacccgtccggaaggtacgctgaaccatctcgctttttgtcgccagcatggcaaagggatggtgatcggcactacggggtttgacgaagccggtaaacaagcaattcgtgacgccgctgccgatattgcgattgtctttgctgccaattttagcgttggcgttaacgtcatgcttaagctgctggagaaagcagccaaagtgatgggtgactacac

SEQ ID NO: 4 - DNA, 1879 bp, Artificial sequence
  hly gene sequence
tagataactgaggatctgaagttcctatactttctagagaataggaacttcggaataggaacttcagatcctcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcctctagaaataattttgtttaactttaagaaggagatatacatatgaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgcctggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaataataactgcag

SEQ ID NO: 5 - DNA, 490 bp, Artificial sequence
  dapB 3′ sequence
ctgaaagattgcgcggtctacagtcgtgaaggccacaccggtgaacgtgtgcctggcaccattggttttgccaccgtgcgtgcaggtgacatcgttggtgaacataccgcgatgtttgccgatattggcgagcgtctggagatcacccataaggcgtccagccgtatgacatttgctaacggcgcggtaagatcggctttgtggttgagtggtaaggaaagcggtctttttgatatgcgagatgtacttgatctcaataatttgtaaccacaaaatatttgttatggtgcaaaaataacacatttaatttattgattataaagggctttaatttttggcccttttatttttggtgttatgtttttaaattgtctataagtgccaaaaattacatgttttgtcttctgtttttgttgttttaatgtaaattttgaccatttggtccacttttttctgctcgtttttatttcatgcaatcttcttgctgcgc

SEQ ID NO: 6 - DNA, 677 bp, Artificial sequence
  dapB 3′ flanking sequence
aagcgttttccagaacaggttagatgatctttttgtcgcttaatgcctgtaaaacatgcatgagccacaaaataatataaaaaatcccgccattaagttgacttttagcgcccatatctccagaatgccgccgtttgccagaaattcgtcggtaagcagatttgcattgatttacgtcatcattgtgaattaatatgcaaataaagtgagtgaatattctctggagggtgttttgattaagtcagcgctattggttctggaagacggaacccagtttcacggtcgggccataggggcaacaggttcggcggttggggaagtcgttttcaatacttcaatgaccggttatcaagaaatcctcactgatccttcctattctcgtcaaatcgttactcttacttatccccatattggcaatgtcggcaccaatgacgccgatgaagaatcttctcaggtacatgcacaaggtctggtgattcgcgacctgccgctgattgccagcaacttccgtaataccgaagacctctcttcttacctgaaacgccataacatcgtggcgattgccgatatcgatacccgtaagctgacgcgtttactgcgcgagaaaggcgcacagaatggctgcattatcgcgggcgataacccggatgcggcgctggcgttagaaaaagcccgcg

SEQ ID NO: 7 - DNA, 5863 bp, Artificial sequence
  Delta dapA omega inv sequence from BM4570
tttcaactcatccatgttatcgcgtaagccgcaggcctgtacggtacagccgggggtcatggctttcgggtagaaataaaccagaacacgctgtccctggaagtcggtcaaattaacttgttctccgtcttgatccggcaagctaaatttcggtgcgatatcaccggctttcagtggattcattacttaactccatcctgttcatcatgttgggaataattgacgacgttaatactgccttgtgcattgagttctgtacatagggctttgaacgcttgctcaatatttgctgcgtccgcagatgcggggctgtgggcggttatctgaatatgcaactgcgcagcccgttcattttcagcaggttgcgtgcgcgacaccagctccgcaatgttcatatgatgcgcgtcgaaaagtgctgtgaagcgttcaattaaatgcggggagtctgccacatcgacctgaacccagacagatgctggcattggcggacgcggacgcgccgtcgtgcgcttcatcacgattaaaagatccagttcggcacctttcaacggtaacgttgattcaatcagagtaatggcattccatgaaccggaaagcagcataataaacgtgaactcttctcccagcatcgccaggcgactgtcttcaatattacagccgcaactactgacatgacgggtgatggtgttcacaattccagggcgatcggcacccaacgcagtgatcaccagataatgttgcgatgacagtgtcaaactggttattcctttaaggggtgagttgttcttaaggaaagcataaaaaaaacatgcatacaacaatcagaacggttctgtctgcttgcttttaatgccataccaaacgtaccattgagacacttgtttgcacagaggatggcccatgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaactagataactgaggatctgaagttcctattccgaagttcctattctctagaaagtataggaacttcagatccctggcaaatattctgaaatgagctgttgacaattaatcatccggctcgtataatgtgtggaattgtgagcggataacaattgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagagaacaacgccgctcaggttaattgagcggcgttgtttttttaaaaggatttgtcgcgataagcgtgagctggcgttaaatgccgatcttacggcccaggatattggcactgcagattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaaagtttagggagatttgatggcttactctgttcaaaagtcgcgcctggcaaaggttgcgggtgtttcgcttgttttattactcgctgcctgtagttctgactcacgctataagcgtcaggtcagtggtgatgaagcctacctggaagcggcaccgcttgcggagcttcatgccccggctggaatgattttgccggtgacctccggtgattatgcaatcccggtgaccaacggtagtggtgctgtcggtaaggcgctggacattcgtccaccagcccagccgctggcactggtttctggcgcgcgtacccagttcacgggcgataccgcttcattgctggtggaaaatggtcgtggcaatactctgtggccgcaggtggttagcgtgctgcaggcgaaaaactacaccatcacccaacgtgatgatgctggtcagacactgaccaccgattgggtacaatggaaccgtctggacgaagacgagcagtatcgtggtcgttatcaaatctctgttaagccgcagggttatcagcaggcggttacggttaaactgctgaacctggaacaggcgggcaaaccggttgcagacgcggcttccatgcagcgttacagcacggagatgatgaacgttatttccgccggtctggataaatctgccactgacgccgcgaacgctgcgcaaaatcgtgcctccaccactatggacgtacaaagtgcagctgatgacaccggtttaccaatgctggtcgtacgcgggccgttcaatgtggtttggcaacgtctgccagcggcgctggaaaaagtgggcatgaaagtgaccgacagcacccgttcgcagggcaacatggccgtaacttataagccgctgtctgacagcgactggcaggaactgggcgcgagcga

SEQ ID NO: 8 - DNA, 993 bp, Artificial sequence
  dapA 5′ flanking sequence
tttcaactcatccatgttatcgcgtaagccgcaggcctgtacggtacagccgggggtcatggctttcgggtagaaataaaccagaacacgctgtccctggaagtcggtcaaattaacttgttctccgtcttgatccggcaagctaaatttcggtgcgatatcaccggctttcagtggattcattacttaactccatcctgttcatcatgttgggaataattgacgacgttaatactgccttgtgcattgagttctgtacatagggctttgaacgcttgctcaatatttgctgcgtccgcagatgcggggctgtgggcggttatctgaatatgcaactgcgcagcccgttcattttcagcaggttgcgtgcgcgacaccagctccgcaatgttcatatgatgcgcgtcgaaaagtgctgtgaagcgttcaattaaatgcggggagtctgccacatcgacctgaacccagacagatgctggcattggcggacgcggacgcgccgtcgtgcgcttcatcacgattaaaagatccagttcggcacctttcaacggtaacgttgattcaatcagagtaatggcattccatgaaccggaaagcagcataataaacgtgaactcttctcccagcatcgccaggcgactgtcttcaatattacagccgcaactactgacatgacgggtgatggtgttcacaattccagggcgatcggcacccaacgcagtgatcaccagataatgttgcgatgacagtgtcaaactggttattcctttaaggggtgagttgttcttaaggaaagcataaaaaaaacatgcatacaacaatcagaacggttctgtctgcttgcttttaatgccataccaaacgtaccattgagacacttgtttgcacagaggatggcccatgttcacgggaagtattgtcgcgattgttactccgatggatgaaaaaggtaatgtctgtcgggctagcttgaaaaaactgattgattatcat

SEQ ID NO: 9 - DNA, 410 bp, Artificial sequence
  dapA 5′ sequence
gtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaac

SEQ ID NO: 10 - DNA, 3374 bp, Artificial sequence
  inv gene sequence
tagataactgaggatctgaagttcctattccgaagttcctattctctagaaagtataggaacttcagatccctggcaaatattctgaaatgagctgttgacaattaatcatccggctcgtataatgtgtggaattgtgagcggataacaattgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagagaacaacgccgctcaggttaattgagcggcgttgtttttttaaaaggatttgtcgcgataagcgtgagctggcgttaaatgccgatcttacggcccaggatattggcactgcag

SEQ ID NO: 11 - DNA, 503 bp, Artificial sequence
  dapA 3′ sequence
attaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaaagtttagggagatttgatggcttactctgttcaaaagtcgcgcctggcaaaggttgcgggtgtttcgcttgttttattactcgctgcctgtagttctgactcacgctataagcgtcaggtcagtggtgatgaagcctacctggaagcggcaccgcttgcggagcttcatgccccggctggaatgattttgccggtgacctccggtgattatgcaatcccggtgaccaacggtagtggtgctgtcggtaaggcgctggacattcgtccaccagcccagccgctggcactggtttctggcgcgcgtacccagttcacgg

SEQ ID NO: 12 - DNA, 583 bp, artificial
  dapA 3′ flanking sequence
gcgataccgcttcattgctggtggaaaatggtcgtggcaatactctgtggccgcaggtggttagcgtgctgcaggcgaaaaactacaccatcacccaacgtgatgatgctggtcagacactgaccaccgattgggtacaatggaaccgtctggacgaagacgagcagtatcgtggtcgttatcaaatctctgttaagccgcagggttatcagcaggcggttacggttaaactgctgaacctggaacaggcgggcaaaccggttgcagacgcggcttccatgcagcgttacagcacggagatgatgaacgttatttccgccggtctggataaatctgccactgacgccgcgaacgctgcgcaaaatcgtgcctccaccactatggacgtacaaagtgcagctgatgacaccggtttaccaatgctggtcgtacgcgggccgttcaatgtggtttggcaacgtctgccagcggcgctggaaaaagtgggcatgaaagtgaccgacagcacccgttcgcagggcaacatggccgtaacttataagccgctgtctgacagcgactggcaggaactgggcgcgagcga